(19) United States
(12) Patent Application Publication
(10) Pub. No.: US 2009/0004677 A1
(43) Pub. Date: Jan. 1, 2009

(54) QUANTITATIVE DETERMINATION OF BCL10

(75) Inventors: Sumit Bhattacharyya, Des Plaines, IL (US); Pradeep K. Dudeja, Darien, IL (US); Joanne K. Tobacman, Burr Ridge, IL (US)

Correspondence Address: AKERMAN SENTERFITT, P.O. BOX 3188, WEST PALM BEACH, FL, 33402-3188, US

(73) Assignee: The Board of Trustees of the University of Illinois, Urbana, IL (US)

(21) Appl. No.: 12/110,796

(22) Filed: Apr. 28, 2008

Related U.S. Application Data
(60) Provisional application No. 60/914,488, filed on Apr. 27, 2007.

(51) Int. Cl. G01N 33/543

(52) U.S. Cl. 435/7.72; 435/7.94; 435/7.92

(57) ABSTRACT

Methods of and kits for quantitatively determining the concentrations of Bcl10 in a biological sample were developed that provide an accurate means of identifying therapeutic molecules with a plurality of therapeutic effects. A solid-phase sandwich enzyme-linked immunosorbent assay (ELISA) for human Bcl10 that provides reproducible, precise measurements was developed and characterized. The sensitivity of the assay is 0.25 ng/ml, enabling accurate detection of small quantities of Bcl10. The sensitive and specific, solid-phase, sandwich ELISA for Bcl10 is well-suited for the accurate determination of Bcl10 values in different experimental conditions, in immune and non-immune cells, and may find use in a clinical context. An ELISA as described herein may have particular clinical utility, since increased Bcl10 is associated with inflammation, infection and malignancy.

### CROSS-REFERENCE TO RELATED APPLICATIONS

The present application claims the priority of U.S. provisional patent application No. 60/914,488 filed on Apr. 27, 2007.

### FIELD OF THE DISCLOSURE

The disclosure relates to the quantitative determination of Bcl10, antibodies, diagnostics, kits and compositions therefore.

### BACKGROUND

As more and more bacteria become antibiotic resistant, it is important to identify a new generation of therapeutic molecules which would replace traditional antibiotic treatments. Furthermore, identification of such drugs which may have other effects such as anti-inflammatories would decrease the amount of drugs on the market providing easier access and less costly drugs. This would be of great importance, especially for senior citizens, and in developing and poorer regions of the world.

### SUMMARY

Bcl10 is involved in a response to inflammation and infection. It is important to determine the amounts of Bcl10 in response to foreign molecules in vivo. Modulation of Bcl10 by different molecules allows for the identification of therapeutic molecules. A quantitative determination of Bcl10, antibodies, diagnostics, kits and compositions are provided.

Lipopolysaccharide (LPS) is recognized as an inducer of the inflammatory response associated with gram-negative sepsis and systemic inflammatory response syndrome. LPS induction proceeds through Toll-like Receptor (TLR) in immune cells and intestinal epithelial cells (IEC). This report presents the first identification of Bcl10 (B-cell CLL/lymphoma 10) as a mediator of the LPS-induced activation of IL-8 in human IEC. Bcl10 is a caspase-recruitment domain (CARD)-containing protein, associated with constitutive activation of NFκB in MALT (mucosa-associated lymphoid tissue) lymphomas. The normal human intestinal epithelial cell line NCM460, normal primary human colonocytes, and ex vivo human colonic tissue were exposed to 10 ng/ml of LPS for 2-6 hours. Effects on Bcl10, phospho-IκBα, NFκB, and IL-8 were determined by Western blot, ELISA, immunohistochemistry, and confocal microscopy. Effects of Bcl10 silencing by siRNA, TLR4 blocking antibody, TLR4 silencing by siRNA, and an IRAK 1/4 inhibitor on LPS-induced activation were examined. Following Bcl10 silencing, LPS-induced increases in NFκB, IκBα, and IL-8 were significantly reduced (p<0.001). Increasing concentrations of LPS were associated with higher concentrations of Bcl10 protein when quantified by ELISA, and the association between LPS exposure and increased Bcl10 was also demonstrated by Western blot, immunohistochemistry, and confocal microscopy. Exposure to TLR4 antibody, TLR4 siRNA, or an IRAK1/4 inhibitor eliminated the LPS-induced increases in Bcl10, NFκB, and IL-8. Identification of Bcl10 as a mediator of LPS-induced activation of NFκB and IL-8 in normal human IEC provides new insight into mechanisms of epithelial inflammation and new opportunities for therapeutic intervention.

Based on the foregoing, a specific and sensitive enzyme-linked immunosorbent assay (ELISA) was developed for precise measurement of Bcl10 in a biological sample. In the experiments described herein, an ELISA of the disclosure precisely measured Bcl10 in small volume cell lysates using recombinant Bcl10 to standardize the assay.

Unless otherwise defined, all technical terms used herein have the same meaning as commonly understood by one of ordinary skill in the art to which this disclosure belongs.

As used herein, “bind,” “binds,” or “interacts with” means that one molecule recognizes and adheres to a particular second molecule in a sample, but does not substantially recognize or adhere to other structurally unrelated molecules in the sample. Generally, a first molecule that “specifically binds” a second molecule has a binding affinity greater than about 10⁵ to 10⁶ moles/liter for that second molecule.

By reference to an “antibody that specifically binds” another molecule is meant an antibody that binds the other molecule, and displays no substantial binding to other naturally occurring proteins other than those sharing the same antigenic determinants as other molecule. The term “antibody” includes polyclonal and monoclonal antibodies as well as antibody fragments or portions of immunoglobulin molecules that can specifically bind the same antigen as the intact antibody molecule.

Although kits, methods and materials similar or equivalent to those described herein can be used in the practice or testing of the present disclosure, suitable methods and materials are described below. All publications, patent applications, patents, and other references mentioned herein are incorporated by reference in their entirety. In the case of conflict, the present specification, including definitions will control. In addition, the particular embodiments discussed below are illustrative only and not intended to be limiting.

### BRIEF DESCRIPTION OF THE DRAWINGS

FIGS. 1A-1B show the increase in Bcl10 following LPS treatment. FIG. 1A is a graph showing NCM460 cells grown in 12-well plates were treated with lipopolysaccharide (LPS) from E. coli 026:B6 (1, 10 and 20 ng/ml) for 6 hours. Cells were assayed for Bcl10 by a solid-phase sandwich ELISA. LPS was found to stimulate Bcl10 protein expression in a dose-dependent manner. Concentrations of Bcl10 were 1.32±0.09 (control), 2.80±0.29, 5.58±0.56, and 8.08±0.82 ng/ml with successive doses of LPS. Statistical significance was determined by one-way ANOVA followed by Tukey-Kramer post-test for multiple comparisons. Differences between the groups, control (Cn) vs. LPS 1, 10 and 20 ng/ml are statistically significant (p<0.001). Data are the mean ±S.D. of three biological and two technical replicates. * designates statistical significance (p<0.001).

FIG. 1B is a scan of a photograph showing the increase in Bcl10 in response to LPS (10 ng/ml) was also detected by Western blot. Whole cell extracts of control and LPS-treated cells (for 6 hours) were separated by SDS-PAGE on a 12% gel. Proteins were transferred to a nitrocellulose membrane and probed with a mouse monoclonal antibody for Bcl10.

FIGS. 2A-2B shows the increased intensity and distribution of Bcl10 in ex vivo human colonic epithelial cells following exposure to LPS by immunohistochemistry.

FIG. 2A: After treatment with LPS 10 ng/ml for two hours, marked increase in intensity and extent of Bcl10 staining (brown) is evident in ex vivo human colonic cell preparations.

FIG. 2B: Control sections were processed simultaneously under similar conditions, but tissue was not exposed to LPS.

FIGS. 3A-3D are scans of photographs showing the confocal images of Bcl10 in human colonic epithelial cells following LPS demonstrate marked increase in Bcl10. Confocal images of NCM460 cells are presented in FIGS. 3A and 3B, and primary human colonic epithelial cells are displayed in FIGS. 3C and 3D. Green fluorescence identifies Bcl10, rhodamine-phalloidin labeled actin is red, and DAPI-labeled nuclei are blue. Marked increase in Bcl10 staining is evident in the LPS-exposed (10 ng/ml for 6 hours) cells (3B and 3D), compared to the unexposed, control cells (3A and 3C) in which green fluorescence is virtually absent. Merged images present the combined Bcl10, actin, and nuclear staining.

FIGS. 4A-4B are graphs showing the decline in IL-8 in human colonic epithelial cells following silencing of Bcl10 by siRNA.

FIG. 4A: IL-8 in the spent medium was assayed by ELISA. Silencing of Bcl10 by siRNA resulted in 50% reduction in the IL-8 response to LPS in the NCM460 cells, declining from a peak value of 2.60±0.16 to 1.38±0.13 ng/ml, with a baseline value of 0.16±10 ng/ml.

FIG. 4B: In primary human colonic epithelial cells, Bcl10 silencing reduced the effect of LPS on IL-8 by 56%, from a peak of 2.05±0.10 ng/ml to 0.98±0.021 ng/ml. Differences are statistically significant (p<0.001, 1-way ANOVA with Tukey-Kramer post-test). Data are the mean ±S.D. of three biological and two technical replicates.

FIG. 4C is a scan of a photograph of a Western blot which demonstrates the effectiveness of silencing Bcl10 by siRNA. siRNA #4 was used for all subsequent experiments.

FIGS. 5A and 5B are graphs showing LPS-induced increase in IκBα phosphorylation in NCM460 cells reduced by Bcl10 siRNA.

FIG. 5A is a graph showing semi-quantitative determinations of phospho-IκBα, by ELISA that detected phosphorylation of Ser32, demonstrate 100% increases over baseline following exposure to LPS. LPS doses of 1, 10, and 20 ng/ml increased phospho-IκBα 2-8 fold.

FIG. 5B is a graph showing NCM460 cells which were exposed to LPS 10 ng/ml for 6 hours, following knockdown of Bcl10 for 24 hours by siRNA. Phospho-IκBα was measured in the cell lysates by ELISA. 50% reduction in the level of phospho-IκBα occurred. Data are the mean ±S.D. of biological and technical replicates. Differences between the groups are statistically significant (p<0.001, 1-way ANOVA with Tukey-Kramer post-test).

FIG. 6 is a graph showing nuclear translocation of NFκB declined following Bcl10 silencing. Semi-quantitative determination of nuclear NFκB (p65) by ELISA demonstrated decline from an LPS-induced increase of 2.97±0.28 fold, to an increase of 1.04±0.14 fold. Results are the mean ±S.D. of three biological and two technical replicates. Differences between the groups are statistically significant (p<0.001, 1-way ANOVA with Tukey-Kramer post-test).

FIGS. 7A-7B are graphs showing the blocking of the TLR4 receptor by neutralizing antibody reduced the LPS-induced stimulation of IL-8 secretion and Bcl10 protein content of NCM460 cells. TLR4 receptors on NCM460 cells were blocked by neutralizing antibody (10 μg/ml or 20 μg/ml) for 1 hour prior to LPS challenge (10 ng/ml) for 6 hours. Control experiments were done with IgG isotype control (10 and 20 μg/ml). Blocking of TLR4 receptors by antibody inhibited the LPS-induced secretion of IL-8 by 84% (FIG. 7A) and Bcl10 by 72% (FIG. 7B). Differences between the groups are statistically significant (p<0.001, one-way ANOVA followed by Tukey-Kramer post-test).

FIGS. 8A-8B are graphs showing the effect of IRAK 1/4 inhibitor on LPS-induced increases in IL-8 and Bcl10. NCM460 cells were pretreated with 50 μM of IRAK-1/4 inhibitor for 2 hours prior to 6 hours LPS exposure. Inhibition of IRAK 1/4 was performed using the inhibitor N-(2-morpholinylethyl)-2-(3-nitrobenzoylamido)-benzimidazole. The LPS-induced increase in IL-8 response declined by 60% (FIG. 8A), and Bcl10 declined by 73% (FIG. 8B). Differences are statistically significant (p<0.001, 1-way ANOVA with Tukey-Kramer post-test), and suggest that IRAK1/4 mediates LPS-induced IL-8 activation upstream of Bcl10.

FIGS. 9A-9B are graphs showing that the higher potency LPS yields proportionate increase in Bcl10, NCM460 cells were exposed to LPS (10 ng/ml×6 hours) from E. coli (500,000 EU/mg LPS) and from Salmonella enterica of greater potency (800,000 EU/mg LPS). IL-8 response (FIG. 9A) increased 1.43-fold, and Bcl10 response (FIG. 9B) increased 1.62-fold.

FIG. 10 is a graph showing that the silencing of Bcl10 does not affect IL-8 response to TNFα, Il-1β, or DSS in the NCM460 cells. In contrast to the declines in IL-8 secretion produced by Bcl10 silencing when IEC were exposed to LPS or λ-carrageenan (CGN), no reductions in IL-8 response occurred with Bcl10 silencing following exposure to TNFα0.1 (ng/ml), IL-1β (10 ng/ml), or DSS (1 μg/ml) for 24 hours.

FIG. 11 is a graph of the composite standard curve of Bcl10 ELISA. Composite standard curve of Bcl10 ELISA plots log of Bcl10 concentration vs. log of optical density. Slope is 0.5 and y-intercept is 0.126 (n=6).

FIG. 12 shows results from a comparison of Bcl10 concentration measurements by Western blot and ELISA following serial dilutions. Panel A is a Western blot of serial dilutions of a Bcl10 sample. Panel B indicates the results of Bcl10 Elisa and compares the expected and experimental results.

FIG. 13 shows results from a comparison between Western blot and ELISA for Bcl10 following stimulation. Panel A is the representative Western blot of Bcl10 in NCM460 cells. Lane 1 is unstimulated, lane 2 is stimulated with LCG 1 μg/ml for 24 hours. Panel B presents the results of densitometry of three Western blots of Bcl10 following stimulation and correction for β-actin. Panel C presents the corresponding quantitative determinations of Bcl10 by ELISA.

FIG. 14 is a graph of ELISA results for samples treated with increasing concentrations of LCG. Samples were treated with a 100-fold range of LCG from 0.1 μg/ml to 10 μg/ml, with corresponding increases in Bcl10 values from 2.80±0.18 to 6.36±0.56 ng/mg protein.

FIG. 15 shows results from a comparison of Western blot and ELISA for Bcl10 in multiple epithelial cell lines. Panel A is a photograph of a Western blot of Bcl10 from several different cell lines, including Caco2, NCM460, normal colonocytes, IB3 and C38 lung cells, and MCF-7 cells. Panel B is a graph showing the results of Bcl10 by Elisa from these different epithelial cells.

### DETAILED DESCRIPTION

Described herein are methods, assays and kits for determining the concentration of Bcl10 in a biological sample from a human. Bcl10 is an important mediator of cellular inflammation. The data described herein indicate a role for Bcl10 in LPS-mediated inflammation in intestinal epithelial cells. The commonly used food additive carrageenan, which is associated with development of intestinal ulcerations, inflammation, and neoplasms in animal models, activates IL-8 through a Bcl10 mediated pathway in human intestinal epithelial cells (Borthakur A, et al. Am J Physiol Gastrointest Liver Physiol. 2006 Nov. 9. Epub). Bcl10-mediated inflammatory pathway in lymphocytes and macrophages indicates a role of potential mediators in these pathways, including NEMO, IRAK1/4, TRAF2/6, TAK1, TAB 1/2, and MALT1. These intermediates are candidates for functional roles in LPS-induced inflammation in the IEC, as well. The data presented herein indicate that one of the important pathways of IL-8 activation induced by LPS in the normal human IEC proceeds through TLR4→IRAK→Bcl10→phpospho-IκBα→NFκB→IL-8. Based on experiments, including Bcl10 Western blots, immunohistochemistry and fluorescent imaging, quantitative determination of Bcl10 by ELISA, and effects of Bcl10 silencing by siRNA, a direct relationship between LPS exposure and Bcl10 and downstream targets of LPS activation was demonstrated, including phospho-IκBα, NFκB and IL-8.

The increases in Bcl10 following LPS were significantly reduced following exposure of the intestinal epithelial cells to either TLR4 neutralizing antibody or to IRAK inhibitor, thereby indicating the responsiveness of Bcl10 to recognized upstream mediators of an LPS-TLR4-induced cascade in immune cells. Until recently, the Bcl10 pathway of NFκB activation had been described only in cells of immune origin.

Bcl10 activation of an inflammatory cascade in human IEC induced by the polysulfated polygalactan carrageenan (Borthakur A, et al. supra) has been shown. McAllister-Lucas et al described a Bcl10-mediated pathway involving CARMA3 and MALT1 in hepatocytes that was activated by angiotensin II (Proc Natl Acad Sci USA, 2007; vol. 104:139-144). Klemm et al indicated that lysophosphatidic acid-induced activation of NFκB in murine embryonic fibroblasts was mediated by Bcl10 and MALT1 (Proc Natl Acad Sci USA 2007; 104(1):134-138). Wang et al identified Bcl10 as a critical mediator in human embryonic 293 kidney cells, in which G protein-coupled receptors activate NFκB (PNAS 2007; 104(1):145-50). They examined other potential mediators by Western blot and found lysophosphatidic acid and endothelin-1 pathways also involved Bcl10. In their study, TNF-α, LPS, or integrin-induced NFκB activation were not affected in Bcl10 deficient cells. The results presented herein are in contrast to their Western blot based findings with regard to LPS. The extensive analysis described herein, which includes quantitative determinations of Bcl10 levels by solid-phase sandwich ELISA, immunohistochemistry, confocal imaging, and effective Bcl10 silencing by siRNA, indicates that in the human intestinal epithelial cells, LPS activation of NFκB and IL-8 is mediated largely by Bcl10. In contrast, it was found that IL-8 activation by TNFα, IL-1β, or dextran-sodium sulfate (DSS) does not involve Bcl10. LPS from E. coli and Salmonella enterica of different potencies was tested and proportionate changes in Bcl10 and IL-8 were found, providing additional quantitative support for the Bcl10 mediation of IL-8 activation by LPS.

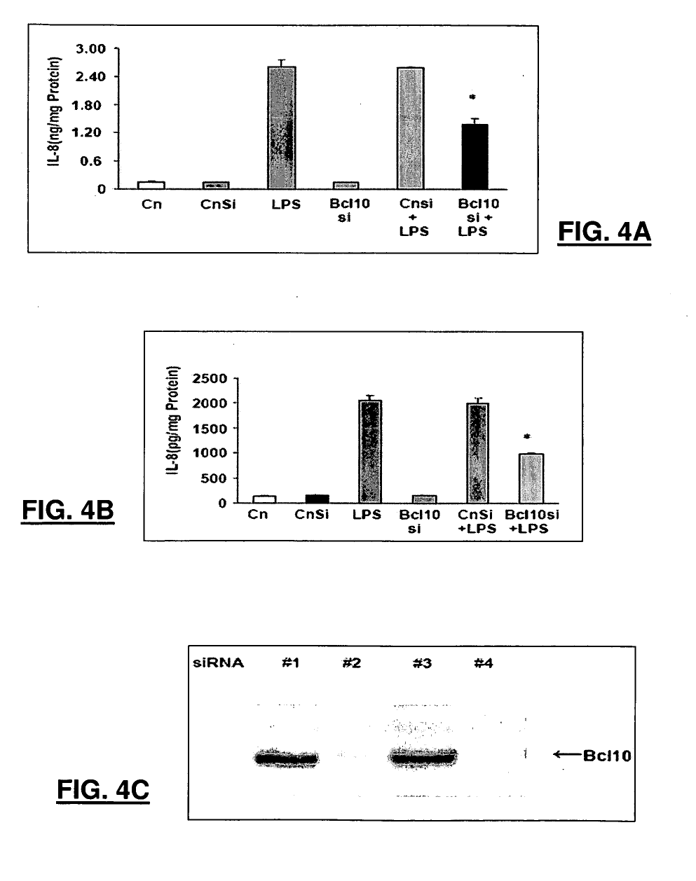

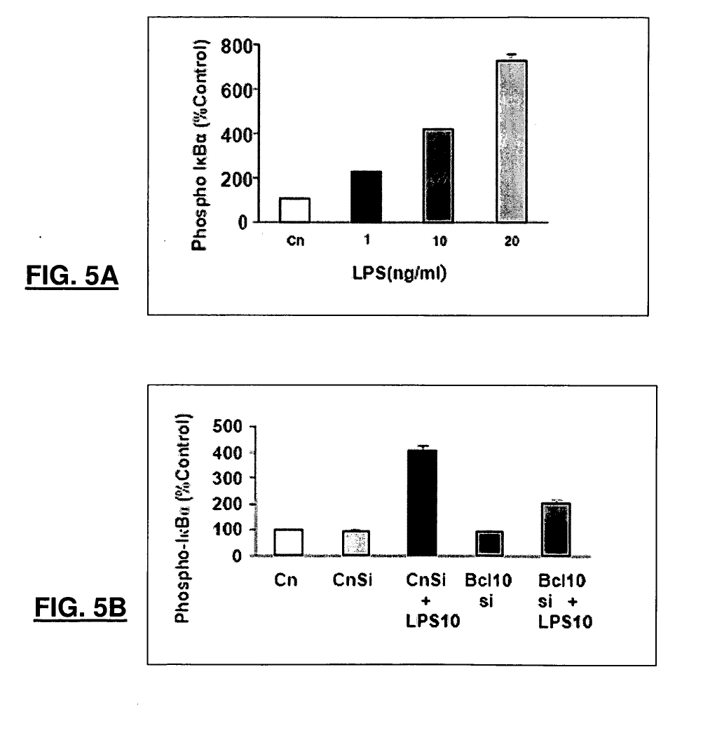

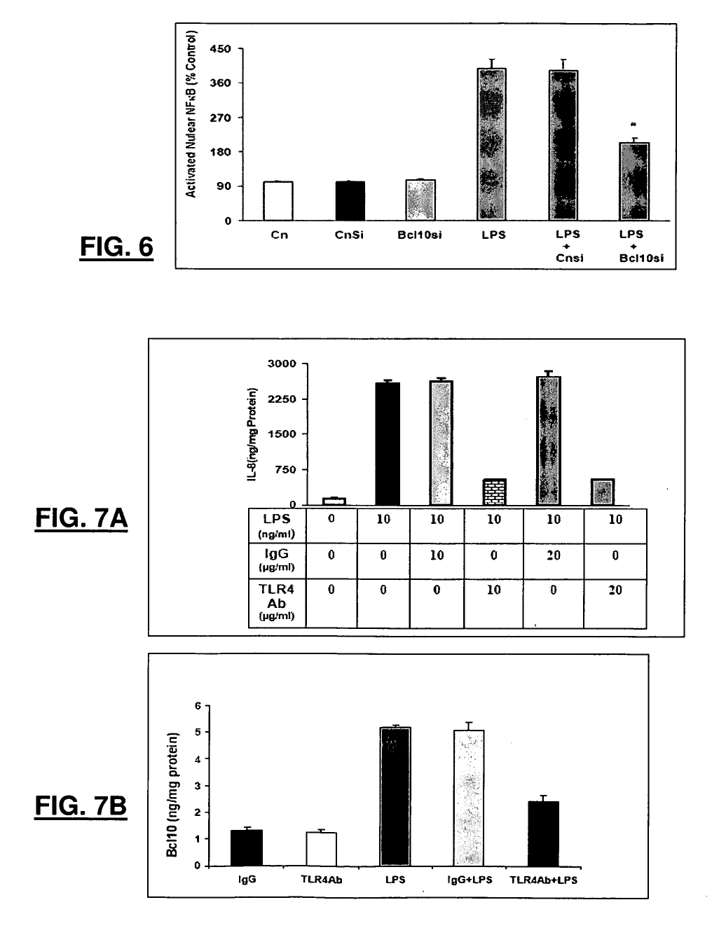

Additional mechanisms of activation appear to be induced by LPS exposure, since Bcl10 silencing does not completely eliminate the increases in phospho-IκBα (FIG. 5B), NFκB (FIG. 6), or IL-8 (FIG. 4A) that followed stimulation by LPS. These additional effects may involve crosstalk with intermediates in the pathway mediated by Bcl10.

Transmission of signals from the hydrophobic lipid A occurs via the leucine-rich TLR4 and via Bcl10. This suggests that the network by which these signals are integrated and transmitted may be sterically mediated by a series of hydrophobic interactions. Bcl10 and NOD2 are both CARD proteins, containing a caspase recruitment domain. The interactions among the different CARD-domain containing proteins, including Bcl10 and the CARMA proteins, which are membrane-associated guanylate kinases, appear to be crucial to the pathway of Bcl10-mediated intracellular inflammation in the immune cells studied previously. The CARD protein has leucinerich repeats and interacts with other CARD proteins, regulating the activation of intracellular signaling mechanisms. Bcl10 has been considered as a potential transcriptional activator and found to act as a mediator of transcriptional activation induced by the transcription factor TFIIB (Liu et al., Biochem Biophys Res Commun 2004, 320:1-6). The nuclear translocation of Bcl10 was stimulated by Akt in association with TNF-α in MCF7 cells (Yeh et al., J Biol Chem 2006, 281:167-175), suggesting the role of Bcl10 in transcription may not be limited to immune cells.

Further consideration of the role of Bcl10 and the interactions among leucine-rich domain-containing proteins may help to explain the mechanism of signal transduction in the intestinal epithelial cells, as well as in immune cells with TLRs. Since LPS-associated inflammation leads to clinically significant illness that is often refractory to the current pharmaceutical interventions, clarification of these signaling mechanisms may lead to therapeutic innovations, as well as improved understanding of the integration of cellular responses to inflammatory stimulation.

The below described embodiments illustrate adaptations of these kits, assays and methods. Nonetheless, from the description of these embodiments, other aspects of the present disclosure can be made and/or practiced based on the description provided below.

### Biological Methods

Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 2003 (with periodic updates). Methods of preparing and using ELISAs are described in treatises including The ELISA Guidebook (Methods in Molecular Biology) by John R. Crowther, 1^{st} ed., Humana Press, Totowa, N.J., 2000; and The Immunoassay Handbook, by David Wild, 3^{rd} ed., Elsevier Science, Cambridge, Mass., 2005.

### Obtaining A Concentration for BCL10 in a Sample

The present disclosure provides a method of obtaining a concentration for BCL10 present in a biological sample from a human. The method includes the steps of providing a biological sample from a human; providing a solid support coated with a first plurality of antibodies against Bcl10; adding the biological sample to the solid support; adding to the solid support a second plurality of antibodies against Bcl10; adding to the solid support a third plurality of antibodies against the second plurality of antibodies resulting in an amount of antibodies bound to antibodies of the second plurality of antibodies, wherein each antibody of the third plurality of antibodies is conjugated to a detectable label; adding to the solid support a substrate which allows for the visualization of the detectable label; measuring the amount of antibodies from the third plurality of antibodies bound to antibodies of the second plurality of antibodies; and comparing the amount to a standard curve to obtain a concentration for BCL10 present in the sample.

The step of providing a biological sample from a human can be performed by conventional methods. A biological sample can be from any site in the body of the human. A biological sample includes a cellular lysate from any suitable fluid or tissue from the human body. For example, a cellular lysate can be prepared from urine, blood, or plasma.

Any antibody that binds to Bcl10 is suitable for use in the present disclosure. In the experiments described herein, a first plurality of antibodies that specifically bind to amino acids 5-19 of human Bcl10 and a second plurality of antibodies that specifically bind amino acids 122-168 of human Bcl10 were used in ELISAs for obtaining the Bcl10 concentration in a biological sample. However, antibodies that recognize any distinct epitope of Bcl10 can be used.

Any suitable detectable label can be used in the methods and kits described herein. In the experiments described below, an enzyme, horseradish peroxidase, was used as the detectable label and TMB was used as the substrate. If an enzyme detectable label is used, any suitable chemiluminescent substrate or chromogenic substrate can be used.

### Kits

The present disclosure includes an ELISA diagnostic kit for measuring the amount of Bcl10 in a sample. A kit includes a solid support having bound thereto a first plurality of antibodies against Bcl10; a second plurality of antibodies against Bcl10; a plurality of antibodies against the second plurality of antibodies, wherein each antibody of the plurality of antibodies against the second plurality of antibodies is conjugated to a detectable label; and an agent for detecting the detectable label. A kit also typically includes at least one standard for establishing a standard curve. A standard for inclusion in the kit is typically a container of recombinant human Bcl10. In the experiments described below, a standard was produced by diluting recombinant human Bcl10 with buffer to concentration of 16 ng/ml, and six additional standards were prepared from baseline 16 ng/ml by serial dilutions. A kit as described herein can include a 96-well microtiter plate as a solid support and a container or vial of 0.2 ml of recombinant human Bcl10 protein at a concentration of 160 ng/ml as a standard. In this example, appropriate amounts of the standard (e.g., 100 μl) can be added in duplicate to the wells of the microtiter plate. Alternatively, any suitable standard can be used.

The following examples are offered by way of illustration, not by way of limitation. While specific examples have been provided, the above description is illustrative and not restrictive. Any one or more of the features of the previously described embodiments can be combined in any manner with one or more features of any other embodiments in the present disclosure. Furthermore, many variations of the present disclosure will become apparent to those skilled in the art upon review of the specification.

All publications and patent documents cited in this application are incorporated by reference in pertinent part for all purposes to the same extent as if each individual publication or patent document were so individually denoted. By their citation of various references in this document, Applicants do not admit any particular reference is prior art.

### EXAMPLES

### Materials and Methods

### Cell Culture of NCM460 Cells and Primary Colonocytes

Non-transformed human colonic epithelial cell line NCM460 were grown in M3:10TM media (INCELL, San Antonio, Tex.), and maintained at 37° C. in a humidified, 5% CO2 environment. Cells were harvested at 60-80% confluency following experimental treatments.

Primary cultures of normal human colonic epithelial cells were initiated from de-identified colon specimens obtained at the time of colectomy through an established protocol, approved by the Institutional Review Board of the University of Illinois at Chicago. Surgical specimens were accessed through the Tissue Bank of the University of Illinois at Chicago Hospitals and Clinics and the Department of Pathology. Patients consented to donate tissue to the Tissue Bank for research purposes. Primary cultures were established as previously described (Borthakur A, et al. Am J Physiol Gastrointest Liver Physiol. 2006 Nov. 9. Epub).

NCM460 and primary colon cells were treated with different concentrations (1, 10 and 20 ng/ml) of lipopolysaccharide (LPS; from E. coli 026:B6, #L2654, Sigma-Aldrich, St. Louis, Mo.) for durations of 2, 4, 6, or 24 hours. The endotoxin level of the LPS from E. coli was reported as 500,000 EU (Endotoxin Units)/mg LPS. The secretion of IL-8 and the cellular content of Bcl10 and phospho-IκBα proteins in response to these treatments were assayed. In the majority of experiments, LPS 10 ng/ml for 6 hours was selected as the optimal dose and duration. A second LPS (from Salmonella enterica serotype Minnesota; #L4641, Sigma-Aldrich, St. Louis, Mo.) was also used for some of the experiments. The endotoxin level of the batch of Salmonella enterica was reported as 800,000 EU/mg LPS.

### Immunohistochemistry of Ex Vivo Colonic Tissue

Immunohistochemistry for Bcl10 was performed on previously frozen, ex vivo, human colonic specimens, after 2 hours of exposure to LPS (10 ng/ml). Control and treated tissue samples were processed simultaneously. Five micron sections were cut by microtome. Tissue sections on the slides were hydrated, and antigen retrieval was performed with DakoTarget Retrieval 10× citrate buffer solution (DakoCytomation) for 20 min in a steamer at 95° C. Slides were then equilibrated to room temperature in the same solution for 20 min, rinsed in dH₂O, placed in buffer solution for 15 min, placed in 3% H₂O₂ for 10 minutes, rinsed in buffer, and protein was blocked for 10 minutes. Slides were incubated with the Bcl10 primary antibody (Santa Cruz Biotechnology) at a 1:50 dilution overnight. Slides were rinsed in buffer, and an anti-mouse secondary antibody (DakoCytomation EnVision mouse monoclonal kit) was applied for 30 minutes. Slides were rinsed in buffer, then treated with 3,3′-diaminobenzidine (DAB) for color detection, rinsed in H₂O, dehydrated, coverslipped with permount, and photographed with Nikon imaging system. No enhancement or modification of the images was performed.

### Confocal Microscopy of Human Intestinal Cells Probed for Bcl10

NCM460 cells and primary human colonic epithelial cells were grown on collagen-coated transwell inserts or 4-chamber tissue culture slides for 24 hours, then treated preparations were exposed to LPS 10 ng/ml for 6 hours. Cells were washed once in 1×PBS containing 1 mM calcium chloride (pH 7.4), fixed for 1.5 hours with 2% paraformaldehyde, then permeabilized with 0.08% saponin. Preparations were washed with PBS, blocked in 5% normal goat serum, incubated overnight with Bcl10 monoclonal antibody (1:100, Santa Cruz) at 4° C., then washed and stained with goat-anti-mouse IgG-FITC (1:100, Invitrogen). Cells were exposed for one hour to phalloidin-Alexa Fluor 568 (Invitrogen) diluted 1:40 to stain actin and to Hoechst 33342 (1:20,000, Invitrogen) for nuclear staining. Preparations were washed thoroughly, mounted, and observed using a Zeiss LSM 510 laser scanning confocal microscope equipped with a 63× water-immersion objective. Excitation was at 488 nm and 534 nm from an Ar/Kr laser and at 361 nm from a UV laser. Green and red fluorescence were detected through LP505 and 585 filters. The fluorochromes were scanned sequentially and images were recorded with Zeiss LSM Image Browser software. Single channel and merged images are presented.

### ELISA for IL-8

The secretion of IL-8 in the spent media of control and treated NCM460 cells and primary colon cells was measured by the DuoSet Enzyme-linked Immunosorbent Assay (ELISA) kit for human IL-8 (R&D Systems Inc, Minneapolis, Minn.), according to the manufacturer's instructions. The IL-8 in the spent media was captured into the wells of a microtiter plate pre-coated with specific anti-IL-8 monoclonal antibody. Immobilized IL-8 was then detected by biotin-conjugated secondary IL-8 antibody and Streptavidin-HRP. Hydrogen peroxide/tetramethylbenzidine chromogenic substrate was used to develop the color and the intensity of color was measured at 450 nm with a reference filter of 570 nm in an ELISA plate reader (SLT, Spectra). The IL-8 concentrations were extrapolated from a standard curve plotted using known concentrations of IL-8. The sample values were normalized with total protein content (BCA™ Protein assay kit; Pierce, Rockford, Ill.) and expressed as pg or ng/mg cellular protein.

### ELISA for Bcl10

The expression of Bcl10 in NCM460 cells or primary colon cells was determined by a solid-phase sandwich ELISA designed to quantify cellular Bcl10. Control or treated cells were lysed in RIPA buffer (50 mM Tris-HCl containing 0.15 M NaCl, 1% Nonidet P40, 0.5% deoxycholic acid and 0.1% SDS, pH 7.4) and the cell extracts were stored at −80° C. until assayed. Bcl10 molecules in the samples or standards were captured in the wells of a microtiter plate pre-coated with rabbit polyclonal antibody to Bcl10 (QED Bioscience Inc., San Diego, Calif.). Immobilized Bcl10 molecules were detected by a mouse monoclonal antibody to Bcl10 (Novus Biologicals, Littleton, Colo.) and goat anti-mouse IgG-horseradish peroxidase (HRP) complex (Santa Cruz Biotechnology, Santa Cruz, Calif.). The peroxidase enzyme activity bound to Bcl10 molecules was determined by chromogenic reaction with hydrogen peroxide/tetramethylbenzidine. Color development due to enzymatic activity was stopped by 2N sulfuric acid, and intensity of the color was measured at 450 nm in ELISA Plate reader (SLT, Spectra). Bcl10 concentrations of the samples were extrapolated from a standard curve derived using known concentrations of recombinant Bcl10 (Calbiochem, EMD Bioscience, San Diego, Carlsbad, Calif.). Sample values were normalized with the total cell protein concentrations determined by BCA™ Protein assay kit (Pierce, Rockford, Ill.).

### ELISA for Phospho-IκBα

Cell extracts were prepared from treated and control NCM460 or primary colon cells by washing with PBS, then harvesting in ice-cold Cell Lysis Buffer (Cell Signaling Technology) that contained 20 mM Tris, pH 7.5, 150 mM NaCl, 1 mM EDTA, 1 mM EGTA, 1% Triton X-100, 2.5 mM Sodium Pyrophosphate, 1 mM β-Glycerophosphate, 1 mM Na₃VO₄, 1 μg/ml leupeptin and 1 mM PMSF. Cells were sonicated twice for 20 seconds, and spun at 13,500 g for 10 min at 4° C. The supernatant (cell lysate) was collected and stored at −80° C. until Phospho-IκBα assay was performed.

Phospho-IκBα was determined by commercially available PathScan Sandwich ELISA (Cell Signaling Technology, Danvers, Mass.), following the manufacturer's instructions. IκBα in the samples was captured in microtiter wells that were coated with monoclonal antibody to IκBα. Captured phospho-IκBα in the wells was detected by a specific rabbit phospho-IκBα antibody that detected Ser32 phosphorylation and was then recognized by an anti-rabbit IgG-HRP. The enzyme activity of bound HRP was determined by adding hydrogen peroxide/tetramethyl benzidine chromogenic substrate. The magnitude of the optical density for the developed color was measured in an ELISA reader at 450 nm, after stopping the reaction with 2N Sulphuric Acid. The intensity of the developed color is proportionate to the quantity of phospho-IκBα in each sample. The sample values were normalized with the total cell protein and expressed as % control.

Silencing of Bcl10 mRNA Expression

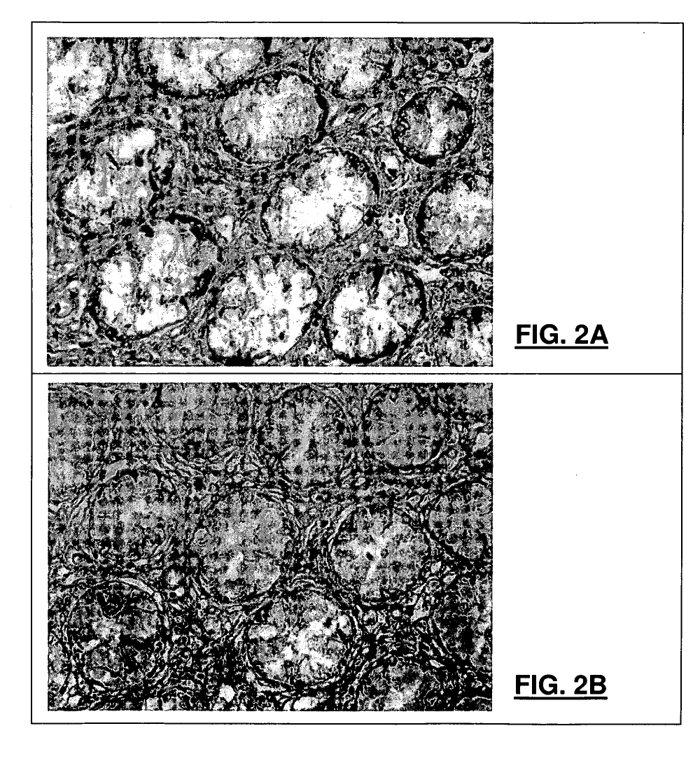

Small interfering (si) RNA for Bcl10 (NCBI NM_{—}003921) silencing and control siRNA labeled with rhodamine were procured (Qiagen, Inc., Valencia, Calif.), and the expression of Bcl10 was silenced in the NCM460 and the primary colon cells with the following protocol. Cells were grown to 60-70% confluency in 6-well tissue culture plate, and on the day of silencing, the medium of the growing cells was replaced with 2.3 ml of fresh growth medium with serum. 0.6 μl/well of 20 μM siRNA (150 ng) was mixed with 100 μl/well of serum-free medium and 12 μl/well of HiPerfect Transfection Reagent (Qiagen). The mixture was incubated for 10 minutes at room temperature to allow the formation of transfection complexes, and then was added dropwise onto the cells. The plate was swirled gently, and then the cells were incubated at 37° C. in a humidified, 5% CO₂ environment. After 24 hours the medium was changed to fresh growth medium. The entry of the transfection complexes into the cells was monitored by observing the fluorescence by microscopy of the control cells that were transfected with rhodamine-tagged control siRNA. The effectiveness of silencing of Bcl10 expression was determined by Western blot of the cell lysates with Bcl10 monoclonal antibody (Santa Cruz Biotechnology, Santa Cruz, Calif.). The blot indicated that siRNA #4 was the most effective, followed by siRNA #2 (FIG. 2, Panel C). Subsequently, siRNA #4 was used in our experiments.

### Neutralization of TLR-4 Receptor by Blocking Antibody

NCM460 cells were grown in 12-well tissue culture plates. At 60-70% confluency, the cells were treated with fresh media containing either 10 μg/ml or 20 μg/ml of TLR4 receptor antibody (BioLegend, San Diego, Calif.) or mouse IgG2a,κ (BioLegend #401502) for one hour, prior to LPS (10 ng/ml) challenge for 6 hours. After 6 hours, the spent media were collected for IL-8 assay, and the cells were harvested for total cell protein determination or Bcl10 assay. Treatment of the NCM460 cells with IRAK 1/4 inhibitor NCM460 cells, grown in 24-well plates, were incubated with 50 μM IRAK1/4 inhibitor (N-(2-morpholinylethyl)-2-(3-nitrobenzoylamido)-benzimidazole (EMD Bioscience, San Diego, Calif.) for 2 hours. After two hours, the media were changed, and new media with or without LPS (10 ng/ml) added. Treatment was terminated at 6 hours, and spent media and cells were collected for IL-8 and other assays.

### Preparation of Whole Cell Lysate and Nuclear Extract

To prepare the whole cell lysate, cells were washed with PBS and harvested.

Complete lysis buffer [in mM: 10 Tris/HCl pH 7.5, 150 NaCl, 5 EDTA, 1 PMSF, 1% triton X-100, 1× protease inhibitor cocktail (Roche Diagnostics, Indianapolis, Ind.)] was added to the cell pellets, and cells were incubated on ice for 30 min with occasional shaking. Then, cells were sonicated twice for 20 seconds and spun at 13,500 g for 20 min at 4° C. The supernatant (cell lysate) was collected and stored at −800 C.

Nuclear extracts were prepared as described previously (Hadjiagapiou C, et al. Am Physiol Gastrointest Liver Physiol 2005; 288:1118-1126). Cells were washed twice with PBS and transferred to an Eppendorf tube. Cells were lysed in lysis buffer [in mM: 10 HEPES (pH 7.9), 10 KCl, 1.5 MgCl₂, 0.1 EDTA, 1 DTT, and 1 PMSF], kept on ice for 10 minutes, and then dounced 10 times on ice in a Dounce homogenizer. The nuclear pellet obtained by centrifugation at 13,000 g for 10 minutes was washed with lysis buffer and resuspended in 50-80 μl of nuclear extraction buffer [in mM: 20 HEPES (pH 7.9), 400 NaCl, 1.5 MgCl₂, 1 EDTA, 1 DTT, 1 PMSF with 25% glycerol] and rotated at 4° C. for 3 hr. Supernatant (nuclear extract) obtained by centrifugation at 13,000 g for 20 minutes was stored at −80° C. until further use.

### Detection of Nuclear NFκB by ELISA

Nuclear extracts were prepared from treated and control NCM460 cells according to the procedure described above. Activated NFκB in the samples was determined by oligonucleotide-based ELISA (Active Motif, Carlsbad, Calif.), following the manufacturer's instructions. Briefly, when the samples were added and incubated in the 96-well microtiter wells, the activated NFκB in the nuclear extracts attached to a consensus nucleotide sequence (5′-GGGACTTTCC-3′ (SEQ ID NO: 1)) that was coated onto the microtiter wells. After washing off the unattached extract, the attached NFκB molecules were captured by antibody to NFκB (p65) and detected by an anti-rabbit-HRP conjugated IgG. Colorimetric readout was performed with hydrogen peroxide/tetramethylbenzidine chromogenic substrate. After stopping the reaction with 2N sulphuric acid, the magnitude of optical density of the developed color was measured in an ELISA plate reader at 450 nm. The intensity of the developed color proportionately represents the quantity of NFκB in each sample. The specificity of the binding of NFκB with the coated nucleotide sequence was determined by comparison to the binding when either free consensus nucleotide or mutated nucleotide was added in the reaction buffer. The sample values were normalized with the total cell protein and expressed as % control.

Western blot of Bcl10

Whole cell lysates or nuclear extracts were separated by SDS-PAGE on a 12% gel. Proteins were transferred to a nitrocellulose membrane (Amersham Biosciences, Piscataway, N.J.) and probed with monoclonal antibody to Bcl10 (Santa Cruz Biotechnology, Santa Cruz, Calif.), followed by IgG-HRP detection antibodies. Immunoreactive bands were visualized on X-ray film (Bio-Rad) using the ECL detection kit (Amersham Biosciences).

### Comparison of Effects of Bcl10 Silencing on TNFα, IL-1β, DSS, LPS, and λ-Carrageenan Activation of IL-8

Bcl10 knockdown was performed as described above in the NCM460 cells, and the effects of TNFα (tumor necrosis factor alpha; 0.1 ng/ml), IL-1β (interleukin-1beta; 10 ng/ml), and DSS (dextran sodium sulfate; 1 μg/ml) exposure for 24 hours on IL-8 secretion were determined by ELISA (R&D) and compared to the effects of LPS (10 ng/ml×6 hours) and CGN (lambda-carrageenan; 1 μg/ml×24 hours).

### Statistical Analysis

Data presented are the mean ±Standard Deviation (SD) of three biological replicates with technical duplicates. Unless stated otherwise, statistical significance was determined by one-way ANOVA, followed by a post-hoc Tukey-Kramer test for multiple comparisons, using GraphPad InStat Software (GraphPad Software, San Diego, Calif.). A p-value of <0.05 is considered statistically significant. Data are expressed as mean ±standard deviation (S.D.). Single asterisk in figures indicates p<0.001.

### Example 1

### Bcl10 Mediates Lipopolysaccharide (LPS)-Induced Activation of NFκB and IL-8 in Human Intestinal Epithelial Cells

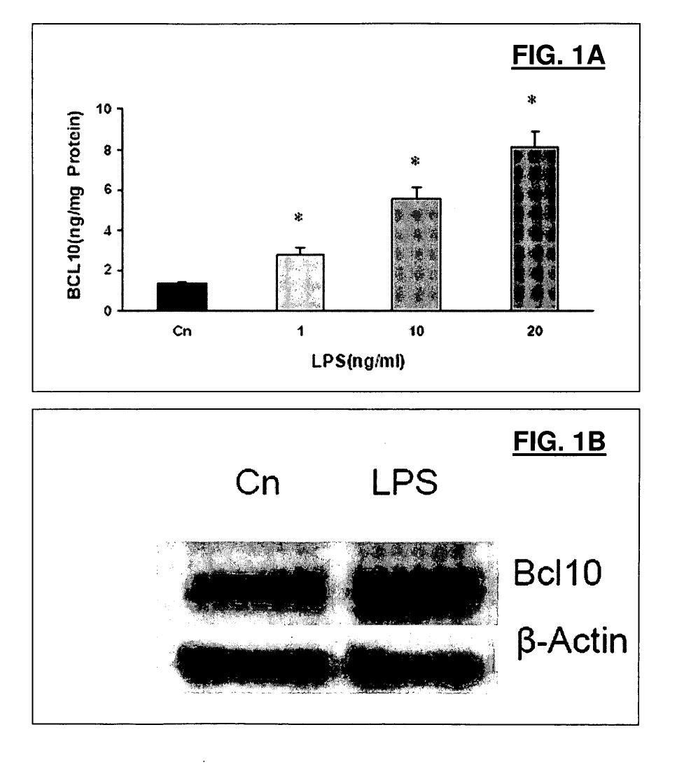

Increase in Bcl10 following exposure to LPS in Intestinal Epithelial Cells: When NCM460 cells were exposed to LPS (1-20 ng/ml for 6 hours), significant increases (p<0.001) in Bcl10 were detectable by Bcl10 ELISA (FIG. 1A). LPS exposures of 1, 10, and 20 ng/ml for 6 hours, produced successive increases in Bcl10 from baseline value of 1.32±0.09 to 2.80±0.29, 5.58±0.56, and 8.08±0.82 ng/ml. Western blot for Bcl10 (FIG. 1B), using a mouse monoclonal antibody (Santa Cruz Biotechnology, Santa Cruz, Calif.) and exposing cells to LPS 10 ng/ml for 6 hours, confirmed the LPS-induced increase in cellular Bcl10 protein.

FIG. 2 presents immunohistochemical staining of Bcl10 in ex vivo sections of normal human colonic tissue obtained from surgery. Increase in intensity and extent of positive (brown) staining for Bcl10 in the tissue that was exposed to LPS (10 ng/ml for two hours) vs. the unexposed control is apparent (Panel A vs. Panel B).

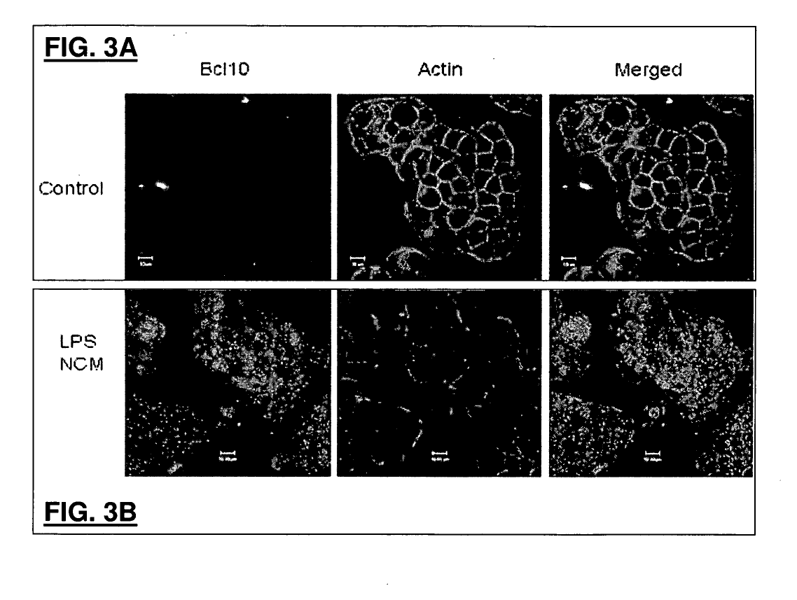

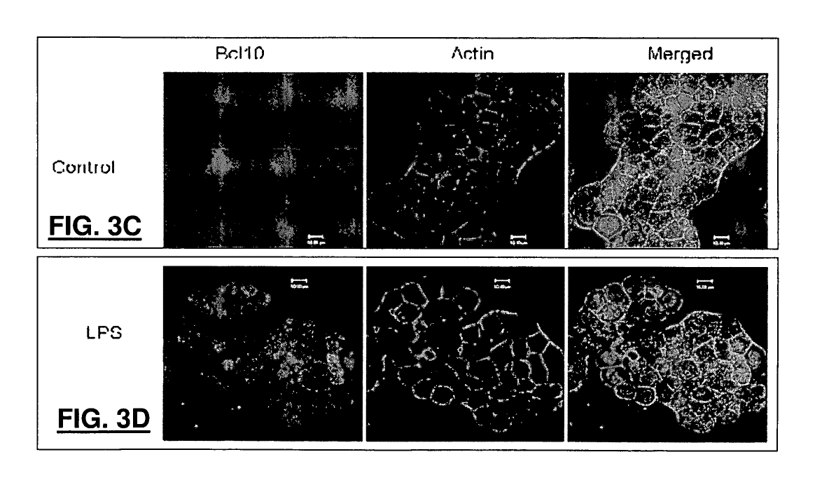

NCM460 cells and primary human colonic epithelial cells were exposed to LPS (10 ng/ml for six hours), then fixed and prepared for confocal imaging. Images are presented in FIG. 3; Bcl10 is tagged with green fluorescence, cell nuclei with blue, and actin fibers with red. The LPS-exposed NCM460 cells (Panel B) and primary colonic epithelial cells (Panel D) have marked increase in staining of Bcl10, compared to the unexposed control cells that have minimal green fluorescence (Panels A and C).

Reduced IL-8 Response to LPS following Bcl10 Silencing by siRNA: Marked increases in IL-8 secretion were observed in response to LPS exposure of the NCM460 cell line (FIG. 4A) and the primary human, colonic epithelial cells (FIG. 4B). LPS doses ranged from 1-20 ng/ml for 6 hours. Following Bcl10 silencing by siRNA, there were significant declines in the LPS-induced responses (p<0.001). Western blot of Bcl10 demonstrates marked decline in Bcl10 following silencing by siRNAs #2 and #4 (FIG. 4C). Greatest silencing effect was by siRNA #4 which was used in subsequent experiments. IL-8 declined by 50% following exposure to Bcl10 siRNA for 24 hours in the NCM460 cells and by 56% in the primary colonic epithelial cells.

Reduced Phospho-IκBα Response to LPS following Bcl10 Silencing: LPS exposure produced marked increase in Phospho-IκBα, measured by Sandwich ELISA kit that detects phosphorylation of Ser32 (FIG. 5A). Following Bcl10 silencing, there was significant reduction (p<0.001) in the LPS-induced increase of phospho-IκBα, with phospho-IκBα declining by 50% from a peak rise to 4.17 times baseline to 2.07 times baseline (FIG. 5B).

Decline in LPS-Induced Activation of NFκB Following Bcl10 siRNA: Nuclear extract of NCM460 cells was prepared and tested for NFκB content, using a 96-well ELISA spotted with oligonucleotide specific for NFκB (p65). Significant decline (p<0.001) in LPS-stimulated nuclear NFκB occurred, following Bcl10 silencing (FIG. 6). NFκB declined from 3.97 times baseline to 2.04 times baseline.

TLR4 Blocking Antibody leads to Reduction of LPS-induced Increase in Bcl10: TLR4 is the cell receptor for LPS and mediates the subsequent LPS-induced inflammatory cascade. Blocking antibody to TLR4 produced a significant reduction (p<0.001) in the LPS-stimulated secretion of IL-8 by the NCM460 cells (FIG. 7A). IL-8 declined 84% from peak value of 2.60±0.05 ng/ml to 0.54±0.01 ng/ml, with baseline control value of 0.14±0.01 ng/ml. When cells were stimulated by LPS following exposure to the TLR4 neutralizing antibody (20 ng/ml×1 hour), there was a statistically significant reduction (p<0.001) in the baseline Bcl10 protein, measured by ELISA (p<0.001) (FIG. 7B). Bcl10 declined to 2.41±0.24 ng/ml, from an LPS-induced peak of 5.20±0.08 ng/ml, with baseline control value of 1.31±0.12 ng/ml, a reduction of 72% in the LPS-induced increase in Bcl10.

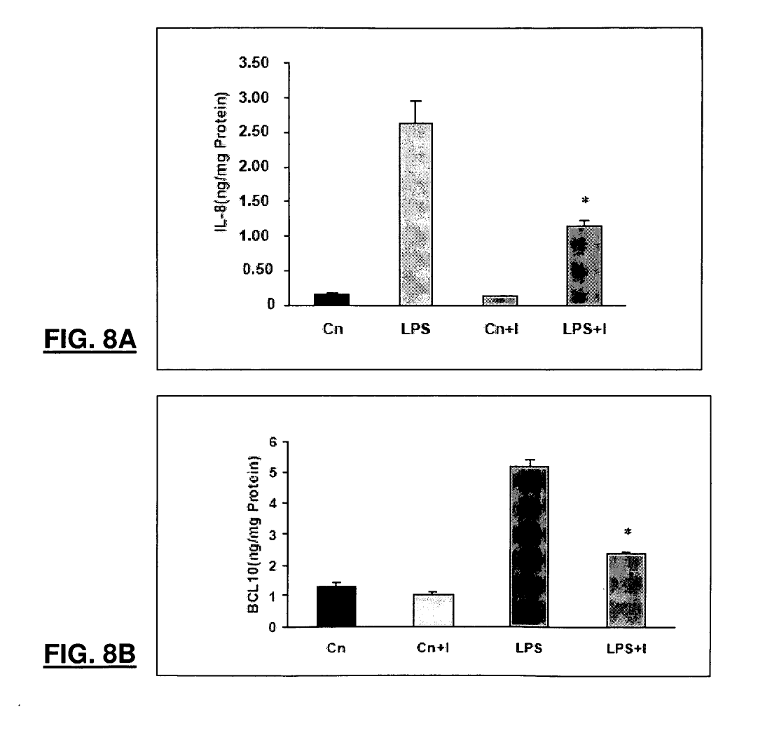

IRAK1/4 Inhibition and Bcl10: Inhibition of IRAK 1/4 was performed using the inhibitor N-(2-morpholinylethyl)-2-(3-nitrobenzoylamido)-benzimidazole. Statistically significant declines in IL-8 (FIG. 8A) and Bcl10 (FIG. 8B) occurred (p<0.001), indicating that IRAK1/4 mediates LPS-induced IL-8 activation and functions upstream of Bcl10. The LPS-induced increase in Bcl10 declined by 73% (from 5.18±0.22 ng/ml to 2.36±0.08 ng/ml), and the IL-8 response declined by 60% (from 2.64±0.31 ng/ml to 1.14±0.08 ng/ml).

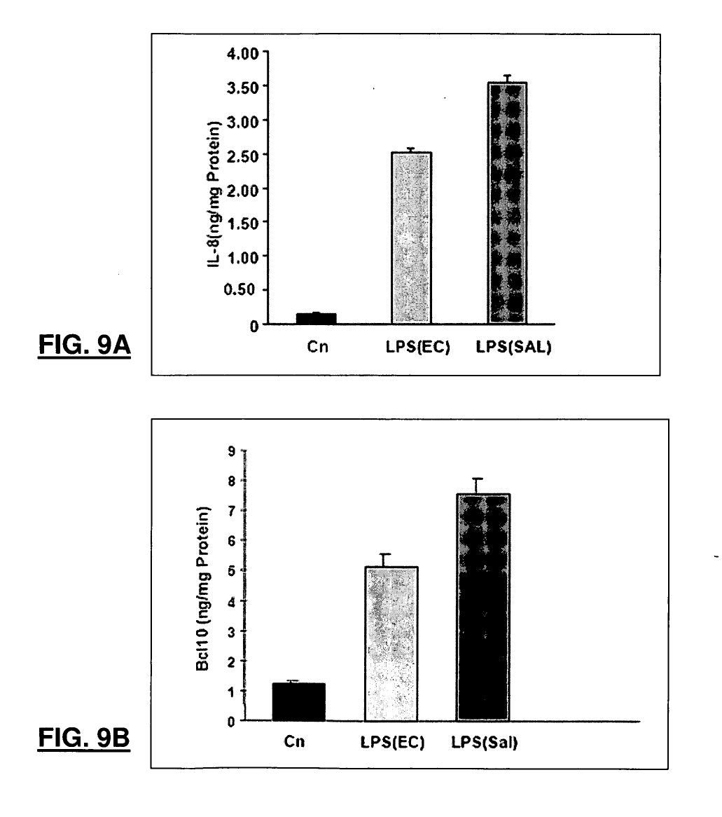

Comparison of Effects of LPS from E. coli and Salmonella enterica: The effects of LPS from E. coli and from Salmonella enterica on the responses of IL-8 and Bcl10 in the NCM460 cells were compared. Results demonstrate IL-8 increase from 2.51±0.06 ng/ml to 3.53±0.11 ng/ml, a 1.4-fold difference with the less potent LPS from E. coli (FIG. 9A). Following exposure to LPS from Salmonella, Bcl10 increased 1.62-fold, consistent with the greater reported potency (800,000 EU/mg LPS vs. 500,000 EU/mg LPS) (FIG. 9B). Salmonella enterica LPS increased Bcl10 from 1.25±0.06 ng/ml to 7.55±0.51 ng/ml, compared to 5.16±0.37 ng/ml from the E. coli LPS. The proportionate changes in LPS potency and Bcl10 level confirm the quantitative association between LPS exposure and Bcl10.

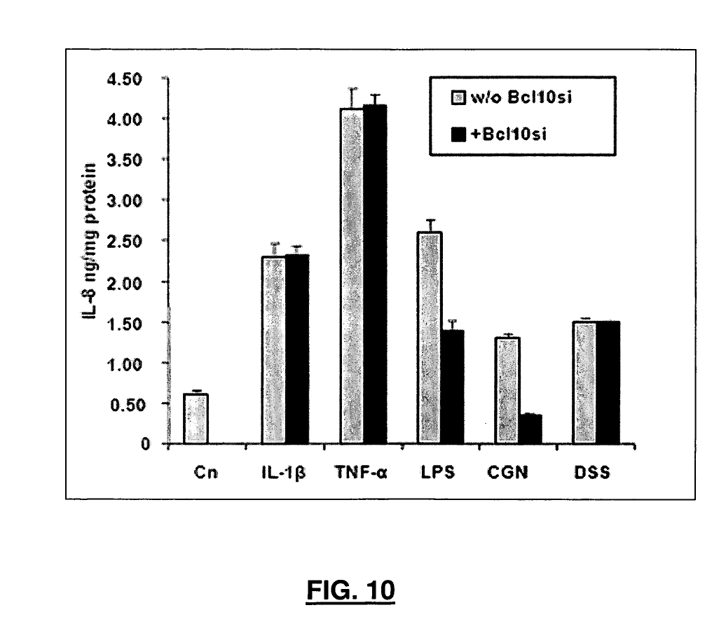

Bcl10 does not mediate IL-8 Activation by TNFα, IL-1β, or DSS: Following Bcl10 silencing by siRNA for 24 hours, NCM460 cells were exposed to TNFα, (0.1 ng/ml), IL-1β (10 ng/ml), DSS (1 μg/ml), or CGN (1 μg/ml) for 24 hours, or LPS (10 ng/ml) for 6 hours (FIG. 10). In contrast, to the marked declines evident with LPS or CNG, Bcl10 silencing had no effect on the IL-8 responses to TNFα, IL-1β, or DSS.

The LPS-induced increases in phospho-IκBα, NFκB, and IL-8 were significantly reduced following silencing of Bcl10 by siRNA. Inhibition of IRAK1/4 and neutralizing antibody for TLR4 produced marked reductions in the LPS-induced increases in Bcl10 and IL-8. However, the reductions in the LPS-induced effects are not complete, suggesting either incomplete inhibition by Bcl10 siRNA, TLR4Ab, or IRAK inhibitor, or the presence of alternate and multiple cellular pathways of IL-8 activation stimulated by LPS. Since the IL-8 responses induced by TNFα and IL-1β are not affected by silencing of Bcl10, interactions between LPS and pathways activated by these cytokines may provide alternative mechanisms of IL-8 activation following exposure to LPS that are not mediated by Bcl10.

### Example 2

### Development, Evaluation and Application of a Highly Sensitive Microtiter Plate ELISA for Human Bcl10 Protein

With the increasing recognition of the role of Bcl10 in mediation of NFκB activation in non-immune cells, as well as in lymphocytes and macrophages, accurate, quantitative measurements of Bcl10 protein have become increasingly important. Due to the importance of Bcl10 in diverse cell types, a solid-phase ELISA was developed to precisely measure Bcl10 in small volume cell lysates, using recombinant Bcl10 to standardize the assay. Standard curve measures Bcl10 from 0.25 ng/ml to 16 ng/ml, with very low intra- and inter-assay variation. Sample dilution and exogenous Bcl10 recovery experiments, comparisons with Western blot, and linear response to increasing doses of known Bcl10 activators confirm the specificity and precision of the ELISA.

Materials and Methods:

Polyclonal rabbit antibody to Bcl10 (QED Biosciences Inc., San Diego, Calif.), recombinant human Bcl10 (Calbiochem, EMD Biosciences, San Diego, Calif.), mouse monoclonal antibody to Bcl10 (Novus Biologicals, Littleton, Colo.) and anti-mouse IgG-HRP conjugate (Santa Cruz Biotechnology, Santa Cruz, Calif.) were obtained from commercial sources. High binding microtiter plates and tetramethylbenzidine (TMB)/H₂O₂ substrate were obtained (R&D Systems, Minneapolis, Minn.). All other chemicals were obtained from Sigma Chemical Co. (St Louis, Mo.).

NCM460 cells (INCELL, San Antonio, Tex.) were grown in specialized M3:TM media as previously described (Borthakur et al., Liver Physiol. 2006, Nov. 9, Epub.; Moyer et al., Vitro Cell. Dev. Biol. Anim. 1996, (32):315-317). Normal human intestinal epithelial cells were obtained from colonic surgeries in accord with a protocol approved by the University of Illinois at Chicago Institutional Review Board as previously reported. Other cell lines, including Caco2, IB3, C38, and MCF-7 cells were obtained from American Type Culture Collection (ATCC, Manassas, Va.) and grown under the recommended conditions.

Microtiter Plate Coating

A polyclonal rabbit antibody to Bcl10 was procured (QED Biosciences Inc., USA) and diluted with PBS (137 mM NaCl, 2.7 mM KCl, 8.1 mM Na₂HPO₄ and 1.5 mM KH₂PO₄ at pH 7.4) to concentrations of 1, 2 and 4 μg/ml for coating the microtiter plate. This antibody recognizes and binds to amino acids 5-19 of human Bcl10 peptide. 100 μl of antibody was added to each well of the microtiter plate (R&D Systems), sealed, and incubated at room temperature (RT) overnight in order to coat the microplate. 4 μg/ml concentration of this antibody was found to work best to capture cellular Bcl10, and was subsequently used for all assays.

Non-Specific Blocking

Following overnight incubation, the excess antibody solution in the plates was aspirated, and the wells were washed three times with 250 μl of Wash Buffer (0.05% Tween 20 in PBS at pH 7.4). After washing, non-specific blocking was performed by adding 250 μl of Blocking Buffer (2% BSA in PBS) to each well and incubating for 1 hour at RT. After blocking, the plate was washed three times with Wash Buffer.

Preparation of the Samples

Cells were washed with ice cold PBS twice, and then the cells were pelleted by centrifugation at 2000 g for 5 minutes and PBS removed. Pellets were resuspended in cell lysate buffer (20 mM Tris-HCl at pH 7.5, 150 mM NaCl, 1 mM EDTA, 1 mM EGTA, 1% Triton X-100, 2.5 mM sodium pyrophosphate, 1 mM Na₃VO₄, 1 mM β-glycerolphosphate, 1 μg/ml leupeptin, 1× protease inhibitor cocktail, and 1 mM phenylmethylsulfonyl fluoride) and incubated on ice for 10 mins. The lysates were sonicated and kept on ice. The samples were centrifuged at 14,000×g at 4° C. for 10 minutes. Supernatant was collected and stored at −80° C. until further use.

Preparation of Standard Curve

Recombinant human Bcl10 was diluted with Reagent Diluent Buffer (20 mM Trizma, 150 mM NaCl, 0.1% BSA and 0.05% Tween 20, pH 7.4) to a concentration of 16 ng/ml. Six additional standards were prepared from baseline 16 ng/ml by serial dilutions (8, 4, 2, 1, 0.5, 0.25 ng/ml). After preparation of the standards, 100 μl of the standards or 100 μl of the samples (cell extracts) was added in duplicate to wells of the microtiter plate. 100 μl of buffer alone was added to the blank wells. The plate was sealed and incubated at 37° C. for 2 hours. At the end of the incubation, the plate was washed three times with Wash Buffer.

Addition of Second Antibody

The mouse monoclonal antibody to Bcl10 is an IgG1 isotype, able to recognize amino acids 122-168 of Bcl10. Antibody was diluted with Reagent Diluent Buffer to a concentration of 1 μg/ml, and 100 μl was added to each well. The microtiter plate was sealed and incubated for 1.5 hours at 37° C. The wells were washed three times with Wash Buffer.

### Detection by Goat Anti Mouse IgG-HRP

Anti-mouse IgG-HRP conjugate (Santa Cruz) was diluted to 1:3000. 100 μl of diluted conjugate was added to each well. The microtiter plate was sealed and incubated for one hour at 37° C. Wells were washed three times.

### Addition of Substrate

TMB/H₂O₂ substrate (R&D Systems) and final substrate solution were prepared by mixing equal volumes of each solution just before use. 100 μl of solution was added to each well. Microtiter plate was sealed and incubated for 20 minutes at RT to develop the colorimetric reaction.

Measurement of Optical Density

The reaction was stopped by addition of 50 μl of stop solution (2NH₂SO₄) to each well. Optical density in the wells was read at 450 nm with a reference filter of 570 nm in an ELISA plate reader (SLT, Spectra). The sample values were normalized with total protein content (BCA™ protein assay kit, Pierce, Rockford, Ill.) and expressed as ng/mg protein.

Calculation of Concentration

The log of optical density of the standards was plotted against the log concentration of the standards to draw a standard curve. The concentrations of the samples were quantified by comparison to the standard curve.

Intra-Assay and Inter-Assay Variations

Three control samples were selected with low, medium, or high Bcl10 values. Values of six replicates in a single assay and in six different assays were determined, in order to assess the intra-assay and inter-assay variations.

Sample Dilution

The effect of matrix dilution on the performance of the Bcl10 ELISA was determined by sample dilution experiments. Three samples, including one high, one medium, and one low, were diluted by 75%, 50% and 25% (buffer:sample=75:25, 50:50 and 25:75). The Bcl10 content in these diluted samples, as well as in the undiluted samples, were determined by ELISA.

Recovery of Exogenously Added Bcl10 (Spiked Samples)

To determine the precision of the developed ELISA, recovery of exogenously added Bcl10 (spike recovery) was performed by mixing known Bcl10 standards (0.5 ng/ml to 4 ng/ml) with two unknown samples (high and low). All samples were assayed by the Bcl10 ELISA.

Measurements of Serial Dilutions by ELISA and Comparison with Western Blot

A cell lysate sample was diluted 1:2, 1:4 and 1:8 in Reagent Dilution Buffer. Bcl10 content in the diluted and undiluted samples was determined by both ELISA and Western Blot. Proteins in the whole cell lysates were separated by SDS-PAGE on a 12% gel, then transferred to a nitrocellulose membrane (Amersham Biosciences, Piscataway, N.J.) and probed with a Bcl10 antibody (Santa Cruz). Immunoreactive bands were visualized using the ECL detection kit (Amersham).

Correlation Between Western Blot and Developed Bcl10 ELISA

NCM460 cells in culture were treated with LCG (1 μg/ml) for 24 hours. At the end of the treatment, the cells were washed three times with PBS and then harvested by scraping. Control or treated cells were lysed in lysate buffer, and then the cell extracts were examined for Bcl10 by either the developed ELISA or by Western blot, as described above. β-actin band density was used as control. Immunoreactive bands were visualized on X-ray film (Bio-Rad Laboratories, Hercules, Calif.) using the ECL detection kit (Amersham). Densitometric analysis of the band intensity from three separate Western blots was compared to the results of the ELISA determinations.

Measurement of Bcl10 in stimulated cells

When the NCM460 cells reached 50-60% confluency, they were treated with a known stimulator of Bcl10, λ-Carrageenan (LCG) (Borthakur et al., Liver Physiol. 2006, Nov. 9, Epub.) The cells were treated with 0.1, 1 or 10 μg/ml of LCG for 24 hours. After treatment, the cells were harvested by scraping and cell lysates prepared, as described above. Bcl10 content in the cell lysates was measured by the ELISA.

Statistical Analysis

Data presented are the mean ±Standard Deviation (SD). Intra- and -inter-assay coefficients of variation were analyzed for known standards, as well as for unknown, quality control samples. At least three biological samples and two technical replicates were performed for each sample. Statistical significance was determined by one-way ANOVA followed by a post-hoc Tukey's test using GraphPad InStat Software (GraphPad Software, San Diego, Calif.). A p-value of <0.05 is considered statistically significant. Pearson correlation coefficients were calculated using the same software.

Results:

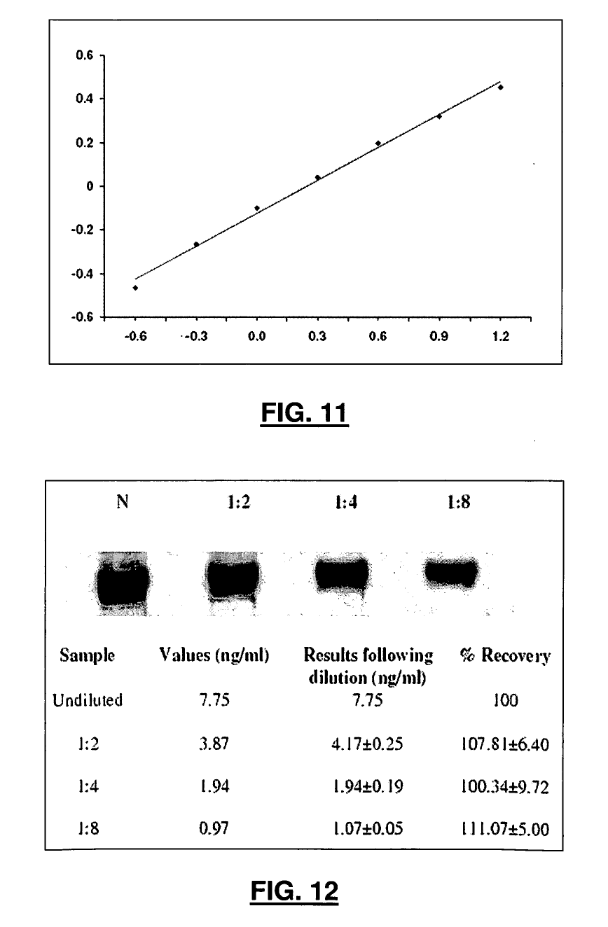

The composite standard curve of six different Bcl10 ELISAs conducted on different days is presented in FIG. 11. The assay range was 0.25 ng/ml minimum to 16 ng/ml maximum. The slope and y-intercept of the curve were 0.5 (0.498±0.039) and −0.126, respectively. The composite standard curve has a sensitivity of 0.25 ng/ml of recombinant human Bcl10 within the 95% confidence limit.

Intra- and inter-assay variations of known standards were calculated by analyzing the optical densities (ODs) of 0.5 ng/ml (low), 2.0 ng/ml (medium) and 8 ng/ml (high) standards of six replicates in the same assay (intra-assay) and of six different assays (inter-assay). Tables 1 and 2 present the intra- and inter-assay variation using the known standards (n=6). Intra-assay coefficient of variation ranged from 2.02% to 4.99%, and inter-assay coefficient of variation was 5.62% to 9.18%.

TABLE 1
Intra-assay Variation of Standards

| | Low Standard | Medium Standard | High Standard |
|---|---|---|---|
| | (0.5 ng/ml) | (2 ng/ml) | (8 ng/ml) |
| Mean (OD) | 0.647 | 1.173 | 2.253 |
| SD | 0.035 | 0.050 | 0.046 |
| CV (%) | 5.384 | 4.297 | 2.052 |

OD = optical density;
SD = standard deviation;
CV = coefficient of variation

TABLE 2
Inter-assay Variation of Standards.

| | Low Standard | Medium Standard | High Standard |
|---|---|---|---|
| | (0.5 ng/ml) | (2 ng/ml) | (8 ng/ml) |
| Mean (OD) | 0.617 | 1.177 | 2.161 |
| SD | 0.057 | 0.104 | 0.121 |
| CV (%) | 9.179 | 8.836 | 5.617 |

OD = optical density;
SD = standard deviation;
CV = coefficient of variation

Intra- and inter-assay variations of the low, medium and high sample values were quantified by the developed ELISA (Tables 3 and 4; n=6). The intra-assay coefficient of variation ranged from 3.31% to 5.74%; and inter-assay coefficient of variation ranged from 6.31% to 9.73%. The low coefficients of variation (<10%) reflect the precision and reproducibility of the developed ELISA.

TABLE 3
Intra-assay Variation of Samples.

| | Low QC | Medium QC | High QC |
|---|---|---|---|
| Mean (ng/ml) | 0.535 | 1.884 | 5.355 |
| SD | 0.027 | 0.108 | 0.177 |
| CV (%) | 5.169 | 5.735 | 3.314 |

QC = quality control samples;
SD = standard deviation;
CV = coefficient of variation

TABLE 4
Inter-assay Variation of Samples

| | Low QC | Medium QC | High QC |
|---|---|---|---|
| Mean (ng/ml) | 0.59 | 1.986 | 5.771 |
| SD | 0.057 | 0.145 | 0.364 |
| CV (%) | 9.727 | 7.296 | 6.314 |

QC = quality control samples;
SD = standard deviation;
CV = coefficient of variation

Three cell extract samples were diluted 75%, 50% and 25% with Reagent Diluent Buffer. Bcl10 content was measured by the ELISA. Table 5 presents the tabulated results of these three samples and their dilutions. Undiluted sample values, as determined in the assay, were used to establish the expected values for subsequent dilutions. Recoveries were calculated as the measured concentration divided by the expected concentration and expressed as percentages. Dilution recoveries ranged from 104% to 109%. All results are statistically significant (two-tailed p-values). Pearson's correlation coefficients (r) are: 1.000 (high, p<0.001), 0.9999 (medium, p=0.0102, and 0.998 (low, p=0.013).

TABLE 5
Bc110 assay results by ELISA following dilution of known sample values

| Sample Value | Dilution | Expected Value | Measured Value | |
|---|---|---|---|---|
| (ng/ml) | Sample:Buffer | (ng/ml) | (ng/ml) | % Recovery |
| 7.148 (high) | 100:0 | | 7.148 ± 0.084 | 100 |
| | 75:25 | 5.361 | 5.611 ± 0.248 | 104.676 ± 4.623 |
| | 50:50 | 3.574 | 3.794 ± 0.033 | 106.153 ± 0.925 |
| | 25:75 | 1.787 | 1.967 ± 0.028 | 110.095 ± 1.566 |
| 3.879 (medium) | 100:0 | | 3.879 ± 0.134 | 100 |
| | 75:25 | 2.909 | 3.05 ± 0.034 | 104.851 ± 1.175 |
| | 50:50 | 1.939 | 2.069 ± 0.082 | 106.685 ± 4.264 |
| | 25:75 | 0.97 | 1.033 ± 0.070 | 106.573 ± 7.277 |
| 1.976 (low) | 100:0 | | 1.976 ± 0.034 | 100 |
| | 75:25 | 1.482 | 1.587 ± 0.066 | 107.087 ± 4.488 |
| | 50:50 | 0.988 | 1.082 ± 0.023 | 109.655 ± 2.313 |
| | 25:75 | 0.494 | 0.539 ± 0.035 | 109.172 ± 7.016 |

Pearson's correlation coefficients between expected ad measured values are: 1.000 (high, p < 0.001), 0.999 (medium, p = 0.0102), and 0.998 (low, p = 0.013).

Two cell extract samples were tested following addition of 0.5 ng, 1 ng, 2 ng and 4 ng of exogenous, recombinant Bcl10 standard. Control “unspiked” samples received an equal volume of Reagent Diluent Buffer. The control sample values, as measured by the assay, were added to the known amount of standard added, in order to establish the expected values for the test “spiked” samples. Recovery was calculated as the measured concentration divided by the expected concentration and expressed as a percentage. Recoveries ranged from 101% to 109% (Table 6). Results are statistically significant. Correlation coefficients (r) are: 0.9993 (high, 95% C.I.=0.9635-1.000, p<0.0007) and 1.0000 (low, 95% C.I.=0.9985-1.000, p<0.0001).

TABLE 6
Recovery of exogenous added Bc110 (“Spike Recovery”) to cell extracts

| Sample | Spiked | Expected | Measured | |
|---|---|---|---|---|
| Value | Value | Value | Value | |
| (ng/ml) | (ng/ml) | (ng/ml) | (ng/ml) | % Recovery |
| 2.121 ± 0.184 | 0.5 | 2.621 | 2.677 ± 0.059 | 108.084 ± 7.120 |
| (high) | 1.0 | 3.121 | 3.250 ± 0.108 | 109.027 ± 4.238 |
| | 2.0 | 4.121 | 4.073 ± 0.086 | 102.356 ± 4.728 |
| | 4.0 | 6.121 | 6.062 ± 0.282 | 101.393 ± 6.172 |
| 0.547 ± 0.007 | 0.5 | 1.047 | 1.105 ± 0.021 | 105.592 ± 2.04 |
| (low) | 1.0 | 1.547 | 1.621 ± 0.036 | 104.79 ± 2.35 |
| | 2.0 | 2.547 | 2.732 ± 0.037 | 107.301 ± 1.478 |
| | 4.0 | 4.547 | 4.928 ± 0.114 | 108.389 ± 2.495 |

Results are statistically significant. Pearson's correlation coefficients between expected and measured are 0.9993 (high, p < 0.0007, two-tailed) and 1.000 (low, p < 0.0001, two-tailed).

Cell lysate with high Bcl10 content was serially diluted 1:2, 1:4 and 1:8. Bcl10 content was then assessed in the diluted and undiluted samples by either Western blot or ELISA (FIG. 12). Width and density of Bcl10 bands in the Western blot were reduced at the higher dilutions (Panel A). Similarly, the ELISA test values declined at higher dilutions (Panel B). The correlation coefficient (r) between the expected and measured ELISA results is 0.9983 (p=0.037, two-tailed t-test).

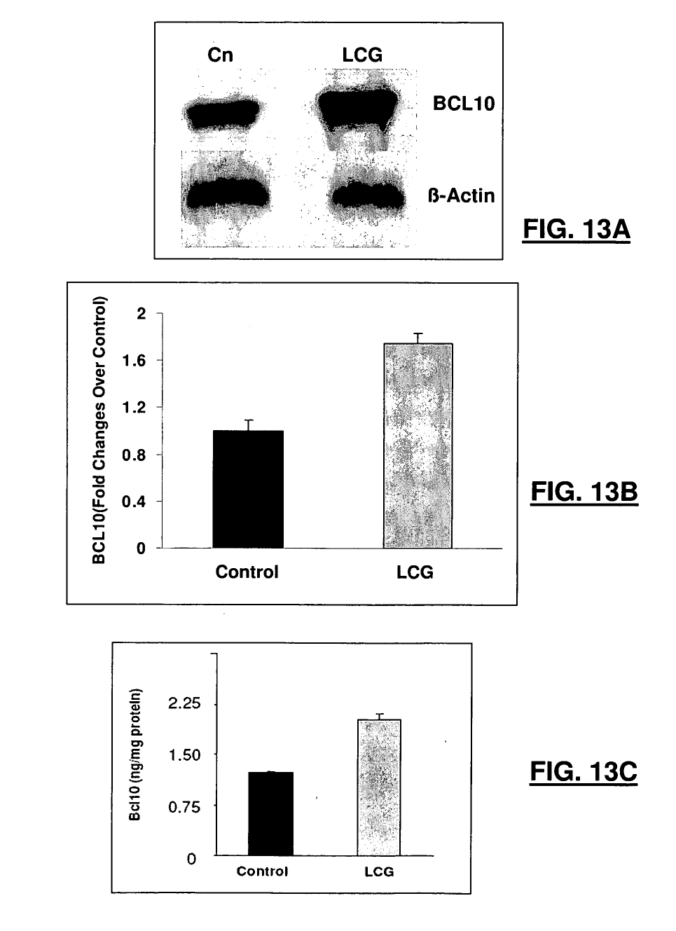

NCM460 cells grown in 12-well plates were treated with λ-carrageenan (LCG) for 6 hours (1 μg/ml). Control or treated cells were lysed in lysate buffer, and the cell extracts were assayed for Bcl10 by Western Blot or by the solid-phase sandwich ELISA (FIG. 13). By Western blot (Panel A), the Bcl10 in treated cells increased 1.75 (LCG) times over control (Panel B). Results of three separate experiments were compared to β-actin and analyzed by densitometry. By ELISA (Panel C), the Bcl10 values increased from control value of 1.22 ng/mg protein to 2.03 ng/mg protein following LCG, a 1.67-fold increase. Correlation coefficient (r) was 0.9972.

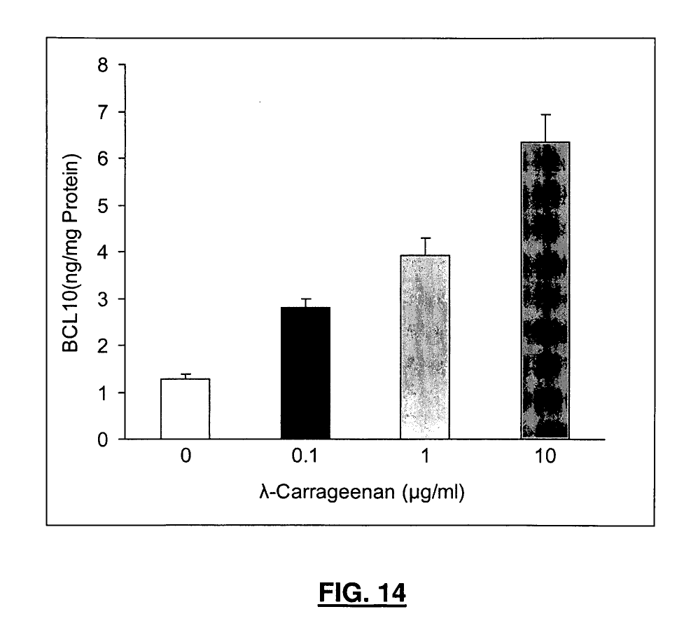

NCM460 cells were exposed to LCG of varying concentrations (0.1 μg/ml, 1 μg/ml, and 10 μg/ml for 24 hours), and Bcl10 values measured. From baseline value of 1.29 (0.09), Bcl10 increased sequentially to 2.80 (0.18), 3.93 (0.37), and 6.36 (0.56) (FIG. 14).

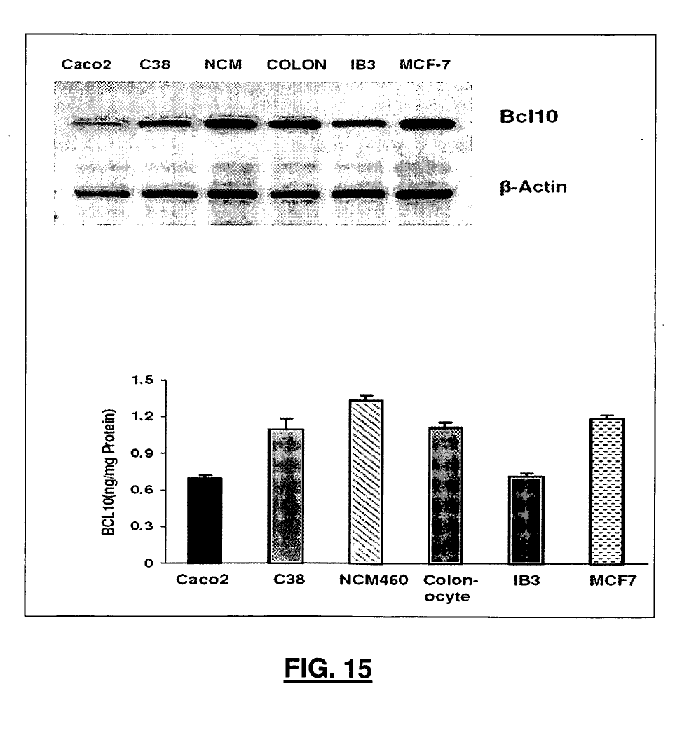

Bcl10 content of several different cell lines was determined by Western blot (FIG. 15, Panel A), and the results were compared to the Bcl10 assay results by ELISA (Panel B). These results identify the presence of Bcl10 in a variety of other epithelial cells, including lung cells and mammary cells.

### Other Embodiments

Any improvement may be made in part or all of the kits and method steps. All references, including publications, patent applications, and patents, cited herein are hereby incorporated by reference. The use of any and all examples, or exemplary language (e.g., “such as”) provided herein, is intended to illuminate the present disclosure and does not pose a limitation on the scope of the invention unless otherwise claimed. Any statement herein as to the nature or benefits of the present disclosure or of the preferred embodiments is not intended to be limiting, and the appended claims should not be deemed to be limited by such statements. More generally, no language in the specification should be construed as indicating any non-claimed element as being essential to the practice of the invention.

The Abstract of the Disclosure is provided to comply with 37 C.F.R. § 1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

What is claimed is:

1. A method comprising:
providing a biological sample from a mammal;
providing a solid support coated with a first plurality of antibodies against Bcl10;
adding the biological sample to the solid support; adding to the solid support a second plurality of antibodies against Bcl10;
adding to the solid support a third plurality of antibodies against the second plurality of antibodies resulting in an amount of antibodies bound to antibodies of the second plurality of antibodies, wherein each antibody of the third plurality of antibodies is conjugated to a detectable label;
adding to the solid support a substrate which allows for the visualization of the detectable label;
measuring the amount of antibodies from the third plurality of antibodies bound to antibodies of the second plurality of antibodies; and
comparing the amount to a standard curve to obtain a concentration for BCL10 present in the sample.

2. The method of claim 1, wherein the first plurality of antibodies against Bcl10 recognize a first epitope and the second plurality of antibodies recognize a second epitope that is different from the first epitope.

3. The method of claim 1, wherein the method comprises a solid-phase sandwich enzyme-linked immunosorbent assay and wherein Bcl10 concentrations in the range of about 0.25 ng/ml to about 16 ng/ml can be obtained.

4. The method of claim 1, wherein the biological sample is selected from the group consisting of: cellular lysate, blood, and urine and wherein the detectable label is horseradish peroxidase and the substrate is selected from the group consisting of: chemiluminescent substrate and chromogenic substrate.

5. The method of claim 1, comprising washing the solid support after the step of adding the sample to the solid support, washing the solid support after the step of adding to the solid support the second plurality of antibodies against Bcl10, and washing the solid support after the step of adding to the solid support the third plurality of antibodies against the second plurality of antibodies.

6. The method of claim 1, wherein the solid support comprises a 96-well microtiter plate.

7. The method of claim 6, wherein the step of measuring the amount of antibodies bound to antibodies of the second plurality of antibodies comprises measuring optical densities in the wells of the 96-well microtiter plate.

8. An enzyme-linked immunosorbent assay diagnostic kit comprising:
a solid support having bound thereto a first plurality of antibodies against Bcl10;
a second plurality of antibodies against Bcl10;
a plurality of antibodies against the second plurality of antibodies, wherein each antibody of the plurality of antibodies against the second plurality of antibodies is conjugated to a detectable label; and
an agent for detecting the detectable label.

9. The enzyme-linked immunosorbent assay diagnostic kit of claim 8, wherein the agent is a substrate.

10. The enzyme-linked immunosorbent assay diagnostic kit of claim 9, wherein the detectable label is an enzyme and the substrate is selected from the group consisting of: chemiluminescent substrate and chromogenic substrate.

11. The enzyme-linked immunosorbent assay diagnostic kit of claim 10, wherein the enzyme is horseradish peroxidase.

12. The enzyme-linked immunosorbent assay diagnostic kit of claim 8, wherein the solid support is a microtiter plate.

13. The enzyme-linked immunosorbent assay diagnostic kit of claim 8, comprising recombinant human Bcl10 for establishing a standard curve.

14. The enzyme-linked immunosorbent assay diagnostic kit of claim 8, wherein the first plurality of antibodies against Bcl10 recognize a first epitope and the second plurality of antibodies recognize a second epitope that is different from the first epitope.

15. The enzyme-linked immunosorbent assay diagnostic kit of claim 8, comprising at least a first wash solution.

16. The enzyme-linked immunosorbent assay diagnostic kit of claim 8, wherein the first plurality of antibodies against Bcl10 are polyclonal rabbit antibodies and the second plurality of antibodies against Bcl10 are mouse monoclonal antibodies.

17. The enzyme-linked immunosorbent assay diagnostic kit of claim 8, comprising instructions for using the kit for quantification of Bcl10 in a biological sample.

18. A method comprising:
fixing a first plurality of antibodies against Bcl10 to a solid support;
providing a second plurality of antibodies against Bcl10;
providing a third plurality of antibodies against the second plurality of antibodies, wherein each of the antibodies of the third plurality of antibodies is conjugated to a detectable label;
providing recombinant human Bcl10 for establishing a standard curve;
providing an agent for detecting the detectable label; and
combining the solid support to which the first plurality of antibodies are fixed, the second plurality of antibodies, the third plurality of antibodies, the recombinant human Bcl10, and the agent for detecting the detectable label in a kit, resulting in an enyzme-linked immunosorbent assay diagnostic kit for measuring a Bcl10 concentration in a biological sample.

19. The method of claim 18, wherein the first plurality of antibodies against Bcl10 recognize a first epitope and the second plurality of antibodies recognize a second epitope that is different from the first epitope.

20. The method of claim 18, wherein the solid support comprises a 96-well microtiter plate, the detectable label is an enzyme, and the agent for detecting the detectable label is a substrate.


SEQUENCE LISTING (1 sequence)

SEQ ID NO: 1 - DNA, 10 bp, ARTIFICIAL SEQUENCE
  OLIGONUCLEOTIDE
gggactttcc